(19) United States
(12) Patent Application Publication
(10) Pub. No.: US 2008/0039906 A1
(43) Pub. Date: Feb. 14, 2008

(54) Light therapeutic device

(75) Inventors: Sheng Bang Huang, Changhua (TW); Chiu Ling Chen, Hsinchu County (TW); Sun Bin Yin, Hsinchu (TW); Yu Chen Yu, Taipei City (TW)

Correspondence Address: BIRCH STEWART KOLASCH & BIRCH, PO BOX 747, FALLS CHURCH, VA, 22040-0747, US

(73) Assignee: Industrial Technology Research Institute

(21) Appl. No.: 11/905,943

(22) Filed: Oct. 5, 2007

Related U.S. Application Data
(63) Continuation-in-part of application No. 11/074,724, filed on Mar. 9, 2005, now abandoned.

(30) Foreign Application Priority Data
Dec. 30, 2004 (TW) 93141350

(51) Int. Cl. A61N 5/06; A61B 18/18

(52) U.S. Cl. 607/88; 606/13

(57) ABSTRACT

A light therapeutic device for an affected part is provided. The light therapeutic device includes an optical element providing light with at least one specific wavelength for treatment of the affected part. When the optical element contacts the affected part, the optical element has a shape corresponding to the shape of the affected part.

### CROSS REFERENCE TO RELATED APPLICATIONS

This application is a Continuation-In-Part of pending U.S. patent application Ser. No. 11/074,724, filed Mar. 9, 2005 and entitled “Light Therapeutic Device”.

### BACKGROUND OF THE INVENTION

1. Field of the Invention

The invention relates to a light therapeutic device, and in particular to a light therapeutic device with a flexible optical element capable of deforming shapes corresponding to an affected part and providing light of specific wavelengths for treatment.

2. Description of the Related Art

Traditionally, oral tumors have been removed by surgical operation using radiotherapy and chemical therapy. Such an intrusive treatment often causes serious problems for patients, such as speaking, chewing, and swallowing and may even cause patients to lose the sensation of taste. Thus, a treatment method eliminating the described problems of the conventional method is desirable. Photodynamic therapy (PDT) employs certain light sensitive materials exposed under light of specific wavelength to kill cells. With tumor cells able to absorb more light transmitted through certain light sensitive materials than normal cells, PDT can be a more effective treatment for oral tumors than the conventional method. Advantages for PDT such as less surgical pain than radiotherapy or chemical therapy has increased interest for those in the filed. For the past ten years, as new light sensitive materials and laser apparatuses have been developed, medical professionals have paid greater attention to PDT, and relevant organizations in the United States, Europe and Japan have applied PDT to clinical tumor treatment experiments.

The light source of PDT permitted by the FDA is a laser source of single wavelength. With costs of purchase and maintenance of a laser sources relatively high, PDT is not popularly used. However, with light emitting diodes (LEDs) having lower weights, lower costs and reduced power consumption, LEDs can replace the laser source to serve as the light source of a PDT.

U.S. Pat. No. 4,907,132 discloses an optical textile with light emitting from selected area for treatment. U.S. Pat. No. 3,508,589 discloses a luminous textile woven by optical fibers. By using some proper method, light can be emitted from the optical fibers. U.S. Pat. No. 6,096,066 discloses a flexible path with a plurality of light sources mounted in spaced-apart array on its undersurface and covered with an optically transparent polymer material. U.S. Pat. No. 4,761,047 discloses a light rays radiation cloth for medical treatment comprising a flexible basic sheet plate, a large number of optical fibers arranged on the surface of the basic sheet plate, and a light rays connector for supplying light rays to the optical fibers. U.S. Pat. No. 5,187,765 discloses a backlighted panel comprising an optical assembly which is disposed in a frame. All the described patents do not teach the light emitting elements can deforms according to an affected part of human body.

### BRIEF SUMMARY OF INVENTION

A light therapeutic device for treatment of an affected part comprises an optical element providing light with at least one specific wavelength for treatment of the affected part. When the optical element contacts the affected part, the optical element has a shape corresponding to the shape of the affected part.

The optical element comprises a flexible optical conductor, which conforms to the affected part when the flexible optical conductor contacts the affected part. The flexible optical element can be made of silicone, silica gel, gum, rubber, or polymer.

The optical element further comprises at least one light source disposed in the flexible optical conductor. The light source comprises light emitting diodes, laser diodes, lamps, cold cathode fluorescent lamps or OLEDs.

In another embodiment, the optical element further comprises an optical textile disposed in the optical conductor, the optical textile is woven by a plurality of optical fibers, whereby light is directed through to, from a light source outside of the optical element.

In another embodiment, the optical conductor has a shape corresponding to a specific part of a body on which the affected part is formed.

A detailed description is given in the following embodiments with reference to the accompanying drawings.

### BRIEF DESCRIPTION OF DRAWINGS

The present invention can be more fully understood by reading the subsequent detailed description and examples with references made to the accompanying drawings, wherein:

FIG. 1 depicts an embodiment of a light therapeutic device of the invention;

FIG. 2 depicts another embodiment of the light therapeutic device of the invention;

FIG. 3 depicts another embodiment of the light therapeutic device of the invention;

FIG. 4 depicts another embodiment of the light therapeutic device of the invention;

FIG. 5 depicts another embodiment of the light therapeutic device of the invention;

FIG. 6 depicts another embodiment of the light therapeutic device of the invention; and

FIGS. 7 and 8 depict another embodiment of the light therapeutic device of the invention.

### DETAILED DESCRIPTION OF INVENTION

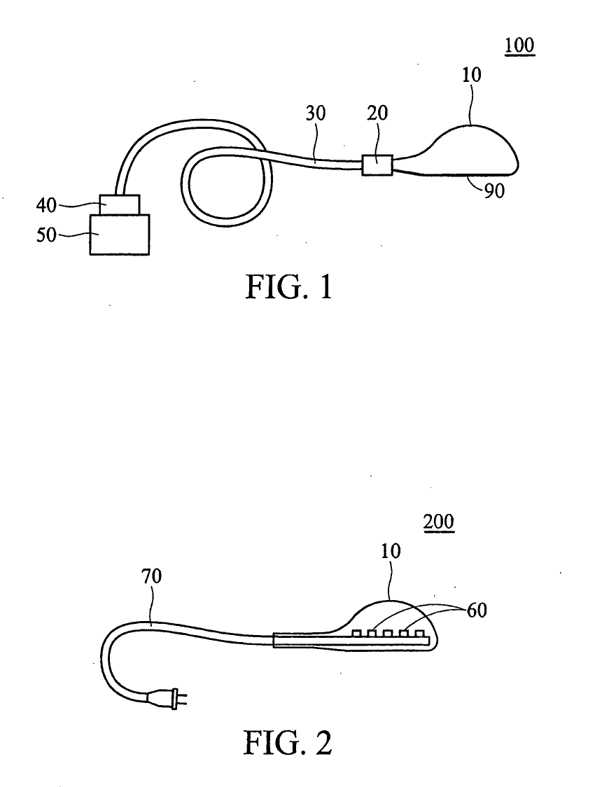

FIG. 1 depicts an embodiment of a light therapeutic device of the invention. The light therapeutic device emits light with specific wavelengths to treat an affected part of a body due to medical conditions, such as oral cancer, cervical cancer or foot tinea. The light therapeutic device 100 comprises an optical element 10, an optical coupler 20, a light guiding tube 30, another optical coupler 40 and a light source 50. The light guide tube 30 is connected to the light source 50 via the optical coupler 40 and connected to the optical element 10 via the optical coupler 20. Light from the light source 50 passes the optical coupler 40, and is directed by the light guiding tube 30 to pass through the optical coupler 20 to enter the optical element 10. In this embodiment, the optical element 10 is an optical conductor which is flexible and capable of conforming to the affected part. The flexible optical conductor can be made of silicone, silica gel, gum, rubber, or polymer. The light source 50 can be light emitting diodes, laser diodes, lamps, cold cathode fluorescent lamps or OLEDs. To enhance the uniformity of light distribution, the optical element 10 can have a plurality of granular objects distributed therein for scattering light entering the optical element. The optical element 10 can also have a reflective layer 90 formed on a part of the optical element 10 to reflect light to the affected part. Since the optical element 10 is flexible, when the optical element is inserted into a mouth for treatment of oral cancer, the optical element 10 conforms to the shape of the oral cavity, whereby the optical element 10 contacts the surface of the oral cavity and has a shape corresponding to the shape of the oral cavity. The light wavelength for treatment of the oral cancer is between 610 nm to 650 nm. When the treatment for oral cancer is performed, a light sensation material is first smeared over the affected part, and then the light therapeutic device is applied to emit light with the wavelengths between 610 nm to 650 nm to the affected part and to activate the sensation material thereby eliminating or suppressing the affected cell.

FIG. 2 depicts another embodiment of the light therapeutic device of the invention. The light therapeutic device 200 comprises a plurality of light sources 60 disposed in the optical element 10. The power for the light source 60 is provided via a cable 70.

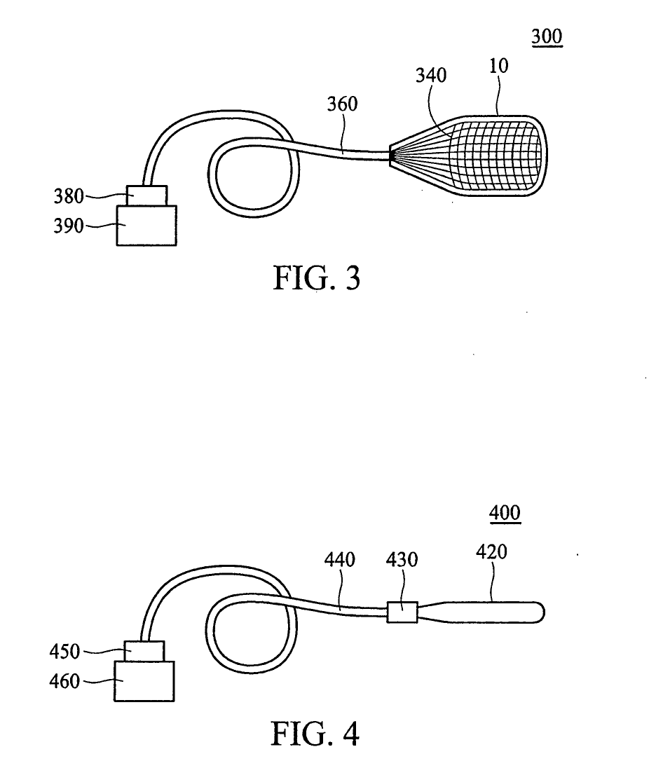

FIG. 3 depicts another embodiment of the light therapeutic device of the invention. The light therapeutic device 300 comprises an optical element 10 and an optical textile 340 disposed in the optical element 10. The optical textile 340 is woven by a plurality of optical fibers. Light from a light source 390 passes through an optical coupler 380 and passes through the optical fibers and finally enters the optical textile. The optical textile 340 is described in the U.S. patent application Ser. No. 11/074,724, filed Mar. 9, 2005.

FIG. 4 depicts another embodiment of the light therapeutic device of the invention. The light therapeutic device 400 comprises an optical element which is rod-shaped or penis-shaped for the treatment of cervical cancer. Light from a light source 460 is directed via an optical coupler 450, a light guide tube 440 and an optical coupler 430, and finally enters the optical element 420. The rod-shaped optical element 420 can be inserted into a vagina or a womb of a female for treating cervical cancer.

The light wavelength for treatment of the cervical cancer is between 610 nm to 650 nm. When the treatment for cervical cancer is performed, a light sensation material is first smeared over the affected part, and then the light therapeutic device is applied to emit light with the wavelengths substantially between 610 nm to 650 nm to the affected part and to activate the sensation material thereby eliminating or suppressing the affected cell.

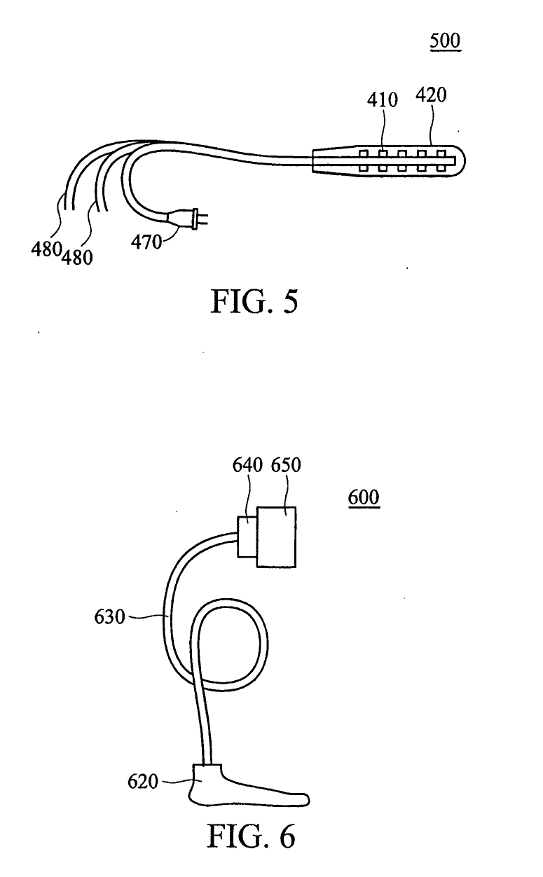

FIG. 5 depicts another embodiment of the light therapeutic device of the invention. The light therapeutic device 500 has a plurality of light sources 410 disposed in the optical element 420. Power is provided to the light sources 460 via a power cable 470. Since the optical element 420 is used in a vagina or a womb, overheating of the light sources 410 may harm parts of the human body. Thus, an inlet tube 480 and an outlet tube 490 for cooling liquid to cool the light sources 410 are provided.

In addition to cervical cancer, the rod-shaped optical element can also be used to treat affected parts in an ear canal or a nasal cavity.

FIG. 6 depicts another embodiment of the light therapeutic device of the invention. The light therapeutic device 600 comprises an optical element 620 which is shoe-shaped or foot-shaped to treat foot tinea. The patient wears the optical element 620 on his/her foot. Light from a light source 650 passes through a light guide element 630 and finally enters the optical element 620.

The light wavelength for treatment of foot tinea is substantially between 360 nm to 420 nm. When the treatment for foot tinea is performed, light sensation material is smeared over the affected part first, and then the light therapeutic device is applied to emit light with the wavelengths between 360 nm to 420 nm to the affected part. In another treatment method, no light sensation material is applied, and the light from the optical element can be directly used to treat the affected part.

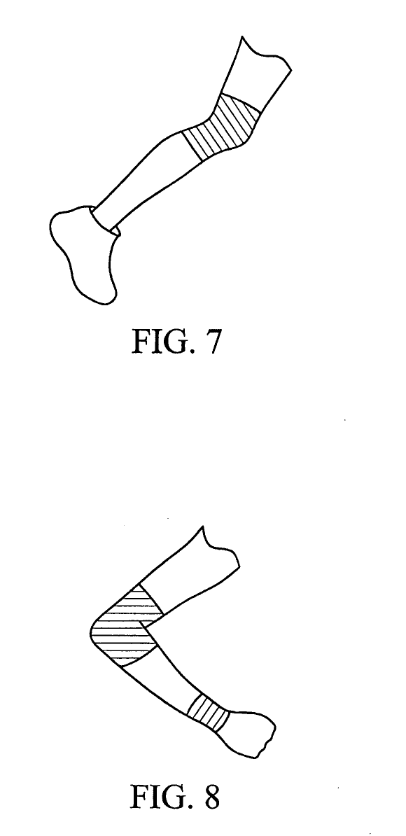

In addition to the shoe-shaped optical element, the optical element can be another shape corresponding to the affected part, for example a glove-shape for hand treatment, or cylinder shaped for knee treatment (FIG. 7) or wrist treatment (FIG. 8). The light wavelength for knee or wrist treatment is substantially between 630 nm to 770 nm.

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A light therapeutic device for an affected part, comprising:
an optical element providing light with at least one specific wavelength for treatment of the affected part, wherein when the optical element contacts the affected part, the optical element has a shape corresponding to the shape of the affected part.

2. The light therapeutic device as claimed in claim 1, wherein the optical element comprises a flexible optical conductor deforming corresponding to the affected part when the flexible optical conductor contacts the affected part.

3. The light therapeutic device as claimed in claim 2 further comprising at least one light source disposed in the flexible optical conductor.

4. The light therapeutic device as claimed in claim 3, wherein the light source comprises light emitting diodes, laser diodes, lamps, cold cathode fluorescent lamps or OLEDs.

5. The light therapeutic device as claimed in claim 2 further comprising an optical textile disposed in the optical conductor, wherein the optical textile is woven by a plurality of optical fibers whereby light is directed to, from a light source outside of the optical element.

6. The light therapeutic device as claimed in claim 2, wherein the flexible optical element comprises silicone, silica gel, gum, rubber or polymer.

7. The light therapeutic device as claimed in claim 1, wherein the optical element comprises a rod-shaped optical conductor inserted into a cavity or channel on which the affected part is formed.

8. The light therapeutic device as claimed in claim 7, wherein the rod-shaped optical conductor comprises silicone silica gel, rubber, resin, plastic, gum, acrylic material or polymer.

9. The light therapeutic device as claimed in claim 7, further comprising at least one light source disposed in the rod-shaped optical conductor.

10. The light therapeutic device as claimed in claim 9, wherein the light source comprises light emitting diodes, laser diodes, lamps, cold cathode fluorescent lamps or OLEDs.

11. The light therapeutic device as claimed in claim 1, wherein the optical element comprises an optical conductor having a shape corresponding to a specific part of a body on which the affected part is formed.

12. The light therapeutic device as claimed in claim 11, wherein the optical conductor comprises silicone, silica gel, rubber, resin, plastic, gum, acrylic material or polymer.

13. The light therapeutic device as claimed in claim 1 further comprising at least one light source disposed in the optical conductor.

14. The light therapeutic device as claimed in claim 13, wherein the light source comprises light emitting diodes, laser diodes, lamps, cold cathode fluorescent lamps or OLEDs.

15. The light therapeutic device as claimed in claim 1 further comprising a light guiding element, whereby light is directed to, from a light source outside of the optical element.

16. The light therapeutic device as claimed in claim 1, wherein the optical element comprises a plurality of granular objects distributed therein for scattering light.

17. The light therapeutic device as claimed in claim 1, wherein the optical element comprises at least one reflective layer formed thereon to reflect light toward the affected part.

18. The light therapeutic device as claimed in claim 1, wherein the light for treatment of the affected part comprises a plurality of wavelengths.

19. A light therapeutic device for oral cancer, comprising:
an optical element comprising an optical conductor having a deformable shape and providing light with at least one specific wavelength for treatment of the affected part, wherein when the optical conductor is inserted into a mouth, the optical conductor deforms to correspond to the shape of the affected part.

20. The light therapeutic device as claimed in claim 19, wherein the optical element further comprises at least one light source disposed in the optical conductor.

21. A light therapeutic device for cervical cancer, comprising:
an optical element having an optical conductor having a penis or a rod-shape for insertion into a vagina or a womb in which the affected part is formed, wherein the optical element provides light with at least one specific wavelength for treatment of the cervical cancer.

22. The light therapeutic device as claimed in claim 21, wherein the optical conductor comprises silicone silica gel, rubber, resin, plastic, gum, acrylic material or polymer.

23. The light therapeutic device as claimed in claim 21, wherein the optical element further comprises at least one light source disposed in the optical conductor.

24. A light therapeutic device for foot tinea, comprising:
an optical element having an optical conductor having a shoe shape or a foot shape surrounding a foot on which the affected part is formed, wherein the optical element provides light with at least one specific wavelength for treatment of the foot tinea.

25. The light therapeutic device as claimed in claim 25, wherein the optical element further comprises at least one light source disposed in the optical conductor.
